(19) United States
(12) Patent Application Publication
(10) Pub. No.: US 2011/0034818 A1
(43) Pub. Date: Feb. 10, 2011

(54) METHOD AND SYSTEM FOR QUANTITATION OF RESPIRATORY TRACT SOUNDS

(75) Inventors: Merav Gat, Nesher (IL); Didi Sazbon, Haifa (IL)

Correspondence Address: THE NATH LAW GROUP, 112 South West Street, Alexandria, VA, 22314, US

(73) Assignee: DEEPBREEZE LTD., Or Akiva (IL)

(21) Appl. No.: 12/936,617

(22) PCT Filed: Apr. 7, 2009
(86) PCT No.: PCT/IL2009/000400

§ 371 Date: Oct. 6, 2010

Related U.S. Application Data
(60) Provisional application No. 61/064,993, filed on Apr. 8, 2008.

(51) Int. Cl. A61B 5/08

(52) U.S. Cl. 600/529

(57) ABSTRACT

Provided is a system and method for analyzing respiratory tract sounds. Sound transducers are fixed on the skin over the thorax that generates signals indicative of pressure waves at the location of the transducer. Processing of the signals involves performing an event search in the signals and determining event parameters for events detected in the search.

### FIELD OF THE INVENTION

This invention relates to medical devices and methods, and more particularly to such devices and methods for analyzing body sounds.

### BACKGROUND OF THE INVENTION

Body sounds are routinely used by physicians in the diagnosis of various disorders. A physician may place a stethoscope on a person's chest or back and monitor the patient's breathing in order to detect abnormal or unexpected lung sounds.

It is also known to fix one or more microphones onto a subject's chest or back and to record lung sounds. U.S. Pat. No. 6,139,505 discloses a system in which a plurality of microphones are placed around a patient's chest. The recordings of the microphones during inhalation and expiration are displayed on a screen, or printed on paper. The recordings are then visually examined by a physician in order to detect a pulmonary disorder in the patient.

U.S. Pat. No. 5,887,208, assigned to the assignee of the present application, discloses a method and system for analyzing respiratory tract sounds in an individual. Transducers are fixed over the thorax. Each transducer generates a signal indicative of pressure waves at the location of the transducer. An acoustic energy signal at each location is then determined from the recorded pressure waves. The acoustic energy signals can be subjected to an interpolation procedure to obtain acoustic energy signals at locations over the thorax where a transducer was not located. The acoustic energy signals at various times over one or more respiratory cycles can be displayed on a screen for viewing and visual analysis.

Chronic obstructive pulmonary disease (COPD) is a lung disease which is manifested clinically by a mid-life onset of slowly progressing symptoms that include chronic cough and sputum production, progressive and persistent dyspnea and wheezing, and is exacerbated by obesity and a long history of smoking. Diagnosis of COPD is typically done by administering a bronchodilator and then determining by spirometery the forced expiratory volume in 1 second (FEV1) and the forced vital capacity (FVC). A post-bronchodilator ratio of FEV1/FVC<0.7 is usually taken as confirmation of an airflow limitation that is not fully reversible, and is thus indicative of COPD. Complete reversibility of airflow is useful in excluding COPD (a rise in FEV1>400 mL).

Asthma is a lung disease in which the airway walls are inflamed and tend to constrict in response to allergens and irritants. Symptoms of asthma include difficulty in breathing, wheezing, coughing, and chest tightness. Sputum production may also be increased.

In contrast to COPD, asthma is an early onset disease of intermittent, reactive symptoms such as episodic wheezing and dyspnea to such triggers as allergies and exercise. Asthma is associated with a family history of the disease. Asthma usually responds to bronchodilators, as determined by post-bronchodilator spirometery. A rise of 12% with an absolute rise in FEV1 of at least 200 mL is considered to be suggestive of bronchoreversibility. Thus, differential diagnosis between COPD and asthma is primarily based on a spirometric test, together with patient history. However, due to significant physiologic overlap in the spirometric data of COPD and asthma patients, bronchoreversibility, as determined by spirometery, does not provide an unambiguous criterion of differential diagnosis of the two diseases. Additional tests, such as a chest X-ray, exhaled nitric oxide levels, and sputum analysis, may be performed to corroborate a diagnosis. However, there is also significant overlap in the patient responses to these tests as well.

### SUMMARY OF THE INVENTION

In the following description and set of claims, two explicitly described, calculable, or measurable variables are considered equivalent to each other when the two variables are proportional to one another.

In its first aspect, the present invention provides a system for analyzing respiratory tract sounds. The system of the invention comprises one or more sound transducers that are configured to be applied to a substantially planar region of the chest or back skin of an individual. Each transducer produces an analog voltage signal indicative of pressure waves arriving to the transducer that is processed by a processor in accordance with the method of the invention.

In one embodiment of the method of the invention, the processor performs an event search of any one of the signals. In another embodiment, the processor is configured to calculate a representative signal by time averaging two or more of the signals and to perform an event search in the representative signal. The processor then determines one or more parameters of the events detected by the event search, such as the time that the events occurred, an intensity of the event, the height of a peak associated with the event, the width of the peak at half the height, half time to rise, half time to fall, or the area under the peak.

In one preferred embodiment, the transducers are divided into two or more sets of transducers. Each set is preferably a contiguous set of transducers in the transducer array and thus overlies a distinct region of the body surface. For example, the transducers may be divided into two sets, one of which consists of one or more transducers overlying the left lung, while the other consists of one or more transducers overlying the right lung. As another example, the transducers may be divided into six sets where the transducers overlying each lung are divided into three subsets (overlying the top, middle and bottom of the lung). For each of the two or more sets of transducers, the processor calculates a representative signal, as explained above and performs an event search on each of the representative signals. The processor then determines one or more parameters of the events detected by the search. The processor may also compare the value of any one or more of the parameters determined for one of the transducer sets with the value of the parameter determined for any one or more of the other transducer sets. For example, the processor may calculate a time delay between the occurrences of corresponding peaks in two sets. The processor may also determine a time delay between repeated occurrences of a particular type of event. The processor may further be configured to calculate a comparison of the values of various event parameters before and after administration of a treatment to the individual. The processor may further be configured to make a diagnosis based upon any one or more of the comparisons. For example, the processor may be configured to diagnose asthma or COPD.

Thus, in its first aspect, the invention provides a system for analyzing sounds in at least a portion of an individual's respiratory tract comprising:
(a) an integer N of transducers, each transducer configured to be fixed on a surface of the individual over the thorax, the ith transducer being fixed at a location xᵢ and generating a signal Z(xᵢ,t) indicative of pressure waves at the location xᵢ; for i=1 to N at times t during a predetermined time interval; and
(b) a processor configured to: receive the signals Z(xᵢ,t) and to process the signals, wherein the processing comprises performing at least one event search; and
   determining one or more event parameters for one or more events detected in an event search.

An event search may be performed on one or more of the signals Z(xᵢ,,t) or on one or more signals P(xᵢ,t) wherein the signals P(xᵢ,t) are obtained after performing one or more procedures on one or more of the signals Z(xᵢ,t) selected from filtering, denoising, smoothing, envelope extraction, and applying a mathematical transformation. Alternatively or additionally, the transducers may be divided into one or more subsets and the processing comprises, for each of one or more of the subsets, calculating a representative signal from one or more of the signals Z(xᵢ,t) or P(xᵢ,t) obtained from transducers in the subset and performing one or more event searches on one or more of the representative signals. The representative signal of a transducer subset may be, for example, a summation or an average signal of the signals obtained by the transducers in the subset.

An event may be, for example, an entire breathing cycle, an inspiratory phase of a breathing cycle, or an expiratory phase of a breathing cycle. The event search may comprise performing any one or more of a peak search, an autocorrelation, a cross correlation with a predetermined function, and a Fourier transform.

One or more of the event parameters may be, for example, a time at which an event occurred, a duration of an event, a magnitude of an event, a height of a peak associated with the event, the width of a peak associated with the event in a signal at half peak height, a half time to rise of a peak associated with the event in a signal, a half time to fall of a peak, an area under a peak; a maximum of the signal during the event, a ratio of a maximum during an inspiratory phase to a maximum during an expiratory phase, a ratio of a duration of an inspiratory phase to a duration of an expiratory phase, and a morphology of a signal during the event.

The processor in the system may be further configured to calculate one or more comparisons between an event parameter value and a predetermined threshold or range of values. The processor may also be configured, for each of one or more pairs of a first representative signal and a second representative signal, to calculate one or more comparisons between an event parameter value calculated for the first representative function and an event parameter value calculated for the second representative function. The processor may be configured to make a diagnosis based upon one or more of the comparisons.

In a preferred embodiment of the invention, the processor is configured to:

(a) determine values of one or more initial event parameters;

(b) determine values of the one or more final event parameters and

(c) compare the values of the initial event parameters to the final event parameters.

In this embodiment, the processor may be configured to make a diagnosis based upon the comparison. The transducers may be divided into one or more sets, and an event parameter is a time at which an event occurred in a representative signal of each set. In this case, the comparison involves determining an extent of synchrony between two signals. Alternatively, or additionally, an event parameter is an average magnitude of a signal over a time period. In this case, the comparison may involve determining a difference in magnitude of two signals obtained during two distinct time periods. The processor may be configured to make a differential diagnosis. Specifically, the processor may be configured to diagnose asthma and/or COPD on the basis of the comparison.

In a most preferred embodiment, the processor is configured to make a differential diagnosis of COPD and asthma wherein :

the one or more initial event parameters are:

(i) an initial mean value of the signal over the predetermined time interval, h₀, calculated for a representative signal obtained on a first subset of transducers prior to administration of a bronchodilator; and

(ii) an initial time delay, Δ̅_{τ}_{₀}, between a time of a peak in a signal calculated for a second transducer set and a time of a corresponding peak calculated for a third transducer set prior to administration of the bronchodilator;

the one or more final event parameters are:
(i) a final mean value of the signal over a predetermined final time interval, h₁, calculated for a representative signal obtained on the first subset of transducers after administration of the bronchodilator; and

(ii) an final time delay, Δ̅_{τ}_{₁}, between a time of a peak in a signal calculated for a second transducer set and a time of a corresponding peak calculated for a third transducer set prior to administration of the bronchodilator;

and wherein the processing comprises:

(a) calculating a change in the mean value of the signal, Δh, where Δh=h₁−h₀;

(b) calculating a change in Δ̅_{τ}, Δ( Δ̅_{τ}), where Δ( Δ̅_{τ})= Δ̅_{τ1}− Δ̅_{τ0};

(c) making a differential diagnosis of COPD if Δ( Δ̅_{τ})>d₁, where d₁ is a predetermined first threshold;

(d) making a differential diagnosis of asthma if (i) Δ( Δ̅_{τ})≦d₁ᵢ; and if (ii) Δ( Δ̅_{τ})<−d₁;

(e) making a differential diagnosis of COPD if (i) |Δ( Δ̅_{τ})|<d₁, and if (ii) Δh≦0;

(f) making a differential diagnosis of COPD if (i) Δh≧0, and if (ii) Δ̅_{τ>d2}, where d₂ is a predetermined second threshold; and

(g) making a differential diagnosis of asthma if (i) Δh≧0, and if (ii) Δ̅_{τ0≦d}_{₂}.

In another of its aspects, the invention provides a method for analyzing sounds in at least a portion of an individual's respiratory tract comprising:

(a) obtaining an integer N of signals Z(xᵢ,t) indicative of pressure waves at locations xᵢ; for i=1 to N over the thorax at times t during a predetermined time interval; and

(b) processing the signals Z(xᵢ,t), wherein the processing comprises performing at least one event search; and

(c) determining one or more event parameters for one or more events detected in an event search.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a system for obtaining an analyzing body sound in accordance with one embodiment of the invention;

FIG. 2 shows a flow chart for carrying out a method of analyzing body sounds in accordance with one embodiment of the invention;

FIG. 3 shows a flow chart of a method for making a differential diagnosis of asthma and COPD in accordance with one embodiment of the invention;

FIG. 4 shows placement of sound transducers over an individual's lungs;

FIGS. 5a, 5b and 5c show signals obtained from a first individual;

FIGS. 6a, 6b and 6c show signals obtained from a second individual;

FIGS. 7a, 7b and 7c show signals obtained from a third individual;

FIGS. 8a, 8b and 8c show signals obtained from a fourth individual;

FIGS. 9a, 9b and 9c show signals obtained from a fifth individual; and

FIGS. 10a, 10b and 10c show signals obtained from a sixth individual.

### DETAILED DESCRIPTION OF PREFERRED EMBODIMENTS

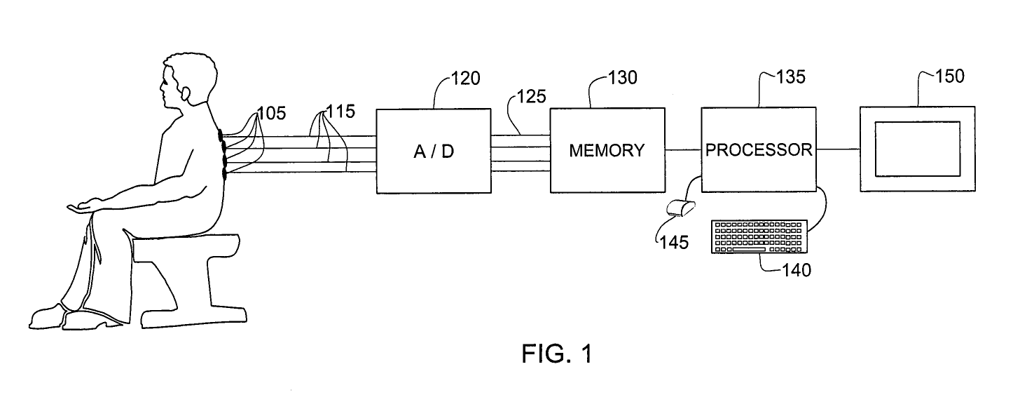

FIG. 1 shows a system generally indicated by 100 for analyzing respiratory tract sounds in accordance with one embodiment of the invention. An integer N of sound transducers 105, of which four are shown, are applied to a planar region of the chest or back skin of individual 110. The transducers 105 may be applied to the subject by any means known in the art, for example using an adhesive, suction, or fastening straps. Each transducer 105 produces an analog signal 115 indicative of pressure waves arriving to the transducer. The analog signals 115 are digitized by a multichannel analog to digital converter 120. The digital data signals Z(xᵢ,t) 125, represent the pressure wave at the location xᵢ of the ith transducer (i=1 to N) at time t. The data signals 125 are input to a memory 130. Data input to the memory 130 are accessed by a processor 135 configured to process the data signals 125. The signals Z(xᵢ,t) 125 may be processed, for example, by filtering, denoising, smoothing, and envelope extraction. The processed signals P(xᵢ,t) may be subjected to a mathematical transformation F to yield transformed signals \tilde{P}(xᵢ,t)=F(P(xᵢ,t)). The signals \tilde{P}(xᵢ,t) may be displayed on a display device 150.

An input device such as a computer keyboard 140 or mouse 145 is used to input relevant information relating to the examination such as personal details of the individual 110. The input device 140 may also be used to input values of the times t₁ and t₂ during which the signals are to be recorded or analyzed. Alternatively, the times t₁ and t₂ may be determined automatically in a respiratory phase analysis of the signals P(xᵢ,t) performed by the processor 135.

In one embodiment of the invention the processor 135 is configured to calculate at least one representative signal R_{S}=R(\tilde{P}(xᵢ,t)) of a subset S of the signals \tilde{P}(xᵢ,t) where For example, R_{S} can be equal to a single signal \tilde{P}(xᵢ,t) or R_{S} can be calculated by time averaging the signals \tilde{P}(xᵢ,t) in the set S. R_{S} may be displayed on the display device 150. The processor is further configured to perform an event search on R_{S}. The event may be, for example, any one or more of a predetermined segment of a respiratory cycle, such as the inspiratory phase, expiratory phase, or a subsegment thereof. An event may be identified by a characteristic morphology in a representative signal R_{S}. For example, an event may be defined by the presence of a peak in a representative signal R_{S} having one or more predetermined characteristics. As additional examples, an event may be identified by a local maximum, local minimum, inflection point, or a derivative of any order or radius of curvature above or below a predetermined value. An event can also be the entire recording. The processor 135 then determines one or more parameters of events detected by the event search, such as the time that the events occurred, the value of a parameter of a peak associated with the event, half time to rise, half time to fall, or the area under the signal during the event, the mean value, maximum or minimum of the signal during the event. The processor 135 may display any one of the representative signals R_{S} or the determined parameters on a display device 150.

In one embodiment, the transducers 105 are divided into two or more sets of transducers. Each set is preferably a contiguous set of transducers in the transducer array and thus overlies a distinct region of the body surface. For example, the transducers may be divided into two sets, one of which consists of one or more transducers overlying the left lung, while the other consists of one or more transducers overlying the right lung. As another example, the transducers may be divided into six sets where the transducers overlying each lung are divided into three subsets (overlying the top, middle and bottom of the lung). For each of the two or more sets of transducers, the processor 135 calculates a representative signal, as explained above and performs an event search on each of the representative signals. The processor then determines one or more parameters of the events detected by the search. The processor 135 may display any one of the parameters on the display device 150. The processor 135 may also compare the value of any one or more of the parameters determined for one of the transducer sets with the value of the parameter determined for any one or more of the other transducer sets for at least one representative signal. For example, the processor may calculate a time delay between the occurrences of corresponding events in two sets between two digital data signals Z(xᵢ,t). Another example, the processor may calculate a time delay between the occurrences of repeat occurrences of an event type within Zₖ(xᵢ,t)

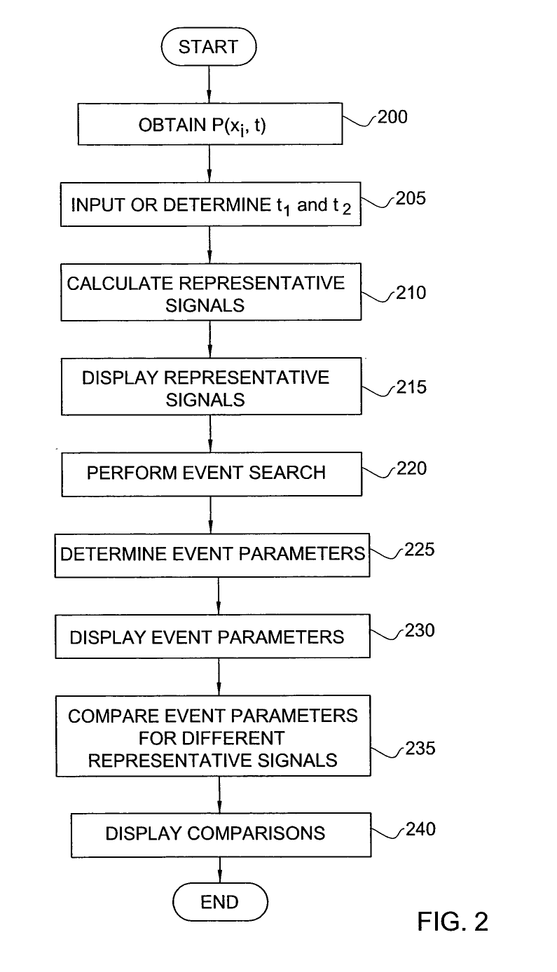

FIG. 2 shows a flow chart for carrying out the method of the invention in accordance with one embodiment. In step 200 the signals Z(xᵢ,t) are obtained from N transducers placed at predetermined locations xᵢ for i from 1 to N on the body surface, where the N transducers may be divided into two or more sets Sᵢ. In step 205 values of t₁ and t₂ are either input to the processor 135 using one or both of the input devices 140 or 145, or are determined by the processor. In step 210, for each transducer set, a representative signal of the transducer set is calculated. In step 215, one or more of the representative signals are displayed on the display device 150. In step 220, for each representative signal, an event search is performed on the representative signal. In step 225, for each representative signal, values of one or more parameters of the events detected in the event search of the signal are determined, such as the times at which the events occurred or the mean value of the representative signal during the event. In step 230, the determined parameter values are displayed on the display device. Finally, in step 235, for each of one or more of the parameters, the values of the one or more parameters determined for each of the representative signals are processed, and in step 240, the results of the processing is displayed on the display device 150.

In one embodiment of the invention, three event types are used, the inspiratory phase, the expiratory phase, and the entire signal over a predetermined time interval. For the events inspiratory phase and expiratory phase, the parameter of the event is the time ti of the peak associated with each occurrence of the event. For the event consisting of the entire signal over the predetermined time interval, the parameter is the mean value h of the signal over the predetermined time interval. For the parameter τ, the processing consists of calculating the time delay Δτ=|τ₁−τ₂|, where τ₁ is the time of a peak in a first representative signal and τ₂ is the time of the corresponding peak in a second representative signal. Δτ is a measure of the extent to which the two representative signals are in synchrony with each other. An average of the Δτ, Δ̅τ, may be calculated if the representative signals cover one or more respiratory cycles.

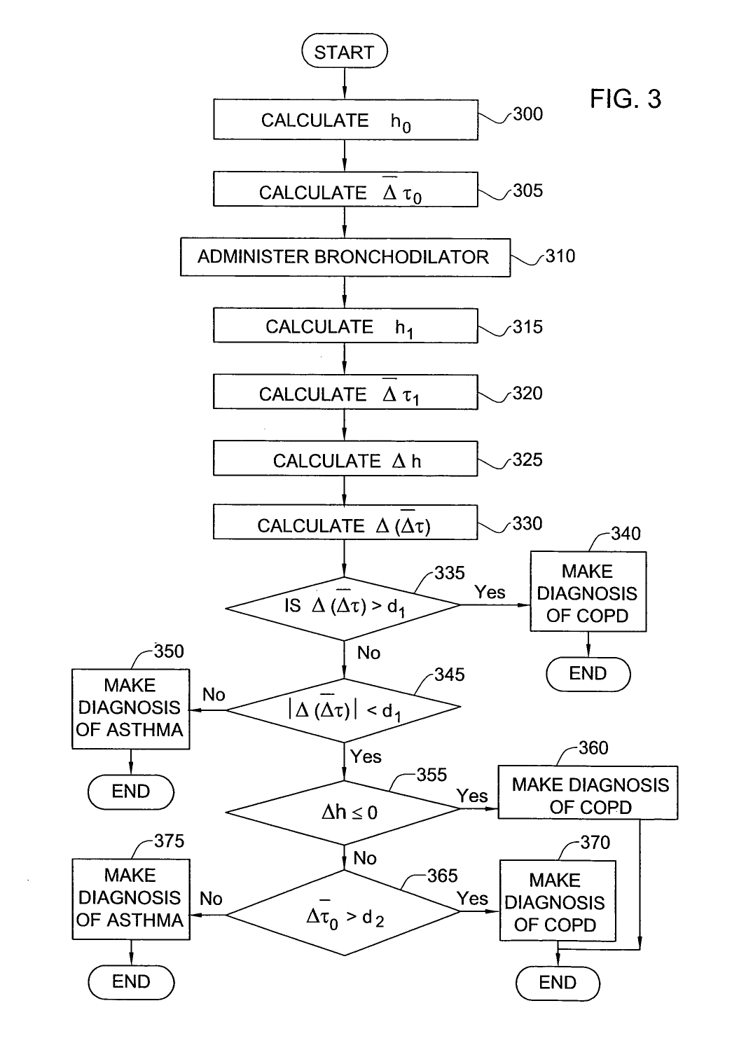

In another of its aspects, the invention provides a method for the differential diagnosis of COPD and asthma. In this aspect of the invention, prior to administration of a bronchodilator, h is calculated for a single representative signal and Δ̅_{τ} is calculated for two representative signals, as explained above. FIG. 3 shows a flow chart for a method of differential diagnosis of COPD and asthma in accordance with this aspect of the invention. In step 300, an initial h, h₀, is calculated as explained above in reference to FIG. 2. In step 305, an initial Δ̅_{τ}, Δ̅_{τ0} is calculated as explained above. In step 310, a bronchodilator is administered to the individual. In step 315, a final h, h₁, is calculated as explained above. In step 320, a final Δ̅_{τ}, Δ̅_{τ1} is calculated as explained above. In step 325, a change in h, Δh, following administration of the bronchodilator is calculated where Δh=h₁−h₀. In step 330, a change in Δ̅_{τ}, Δ( Δ̅_{τ}), following administration of the bronchodilator is calculated where Δ( Δ̅_{τ})= Δ̅_{τ1}− Δ̅_{τ0}.

In step 335, Δ( Δ̅_{τ}) is compared to a predetermined first threshold d₁. If Δ( Δ̅_{τ})>d₁, then the extent of synchrony of the two representative signals decreased as a result of the administration of the bronchodilator, and in step 340 a differential diagnosis of COPD is made, and the process terminates. If at step 335 it is determined that Δ( Δ̅_{τ}) does not exceed d₁, then in step 345 it is determined whether |Δ( Δ̅_{τ})|<d₁. If no (i.e. Δ( Δ̅_{τ})<−d₁), then the extent of synchrony between the two representative signals increased as a result of the administration of the bronchodilator, and in step 350 a differential diagnosis of asthma is made. If at step 345, it is determined that |Δ( Δ̅_{τ})<d₁, then the synchrony of the representative signals did not change significantly as a result of the administration of the bronchodilator, and the process continues with step 355 where the sign of Δh is determined. If Δh<0, then h decreased following the administration of the bronchodilator and in step 360, a differential diagnosis of COPD is made. If at step 355 it is determined that Δh>0, then h increased following administration of the bronchodilator, and the process continues with step 365 where Δ̅_{τ0} is compared to a predetermined second threshold d₂. If in step 365 it is determined that Δ̅_{τ0}>d₂, then in step 370 a differential diagnosis of COPD is made. If in step 365 it is determined that Δ̅_{τ0}≦d₂, then in step 375 a differential diagnosis of asthma is made, and the process terminates.

### EXAMPLES

The system and method of the invention were used for differential diagnosis of COPD and asthma.

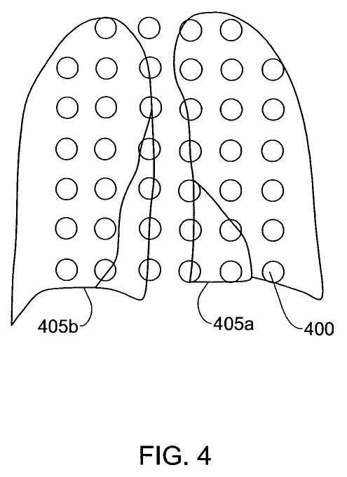

In the cases described below, 40 transducers were placed on a subject's back over the lungs at the locations indicated by the circles 400 in FIG. 4. The curves 405a and 405b show the presumed contours of the subject's left and right lung, respectively. As can be seen, the transducers were arranged in a regular orthogonal lattice with a spacing between the transducers in the horizontal and vertical directions of 5 cm. The signals Z(xᵢ,t) were then recorded over several respiratory cycles. The processing of the signals Z(xᵢ,t) to produce the signal \tilde{P}(xᵢ,t) included band pass filtering between 150 to 250 Hz, envelope extraction and conversion to decibels relative to the saturation level of the transducer. For the parameter τ, the transducers were divided into two sets of 20 transducers. One set, referred to herein as “the left set of transducers” consisted of the transducers overlying the left lung which are shown in FIG. 4 within the contour 405a. The other set, referred to herein as “the right set of transducers” consisted of the transducers overlying the right lung which are shown in FIG. 4 within the contour 405b. A representative signal was calculated for each of the two sets of transducers as the mean of the signals \tilde{P}(xᵢ,t) obtained by the transducers in the set. For the parameter h, the entire set of 40 transducers was used as a single set of transducers, and a representative signal was calculated as the mean of the signals \tilde{P}(xᵢ,t) obtained by the transducers in this set.

Representative signals were obtained before administration of a bronchodilator, and an initial average AT of the two representative signals, Δ̅_{τ0}, was calculated, together with an initial h₀ as explained above. A 2.5 mg dose of the bronchodilator albuterol was then administered to the subject via a nebulizer. 15 min after administration of the bronchodilator, a final Δ̅_{τ1} and h₁ were calculated. The change in the At following administration of the bronchodilator, Δ( Δ̅_{τ}), was also calculated, as was the change in h, Δh.

### Case 1

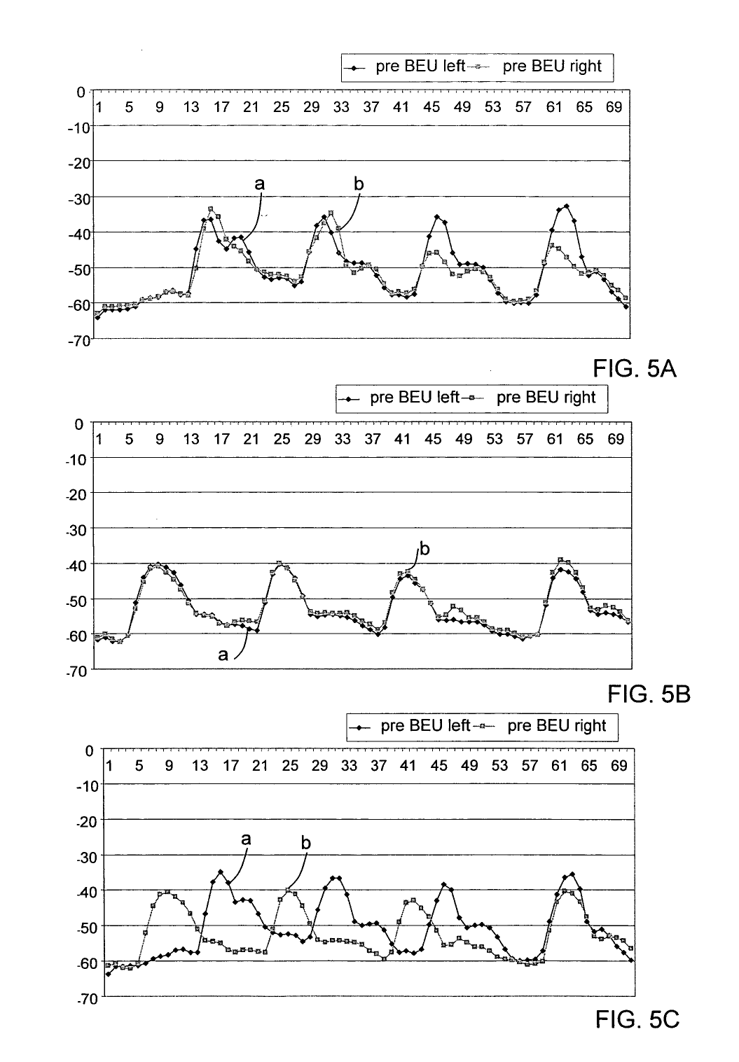

FIG. 5a shows the representative signal obtained as above for the left lung (curve a) and the right lung (curve b) of a subject obtained prior to administration of the bronchodilator. FIG. 5b shows the representative signal obtained as above for the left lung (curve a) and the right lung (curve b) of a subject obtained after administration of the bronchodilator. FIG. 5c shows the average acoustic level in decibels of both lungs before (curve a) and after (curve b) administration of the bronchodilator.

The results are summarized in Table 1.

TABLE 1

| | Δ̅τ0 | Δ̅τ1 | h₀ | h₁ |
|---|---|---|---|---|
| | [# of frames] | [# of frames] | [db] | [db] |
| Patient # 58 | 0.75 | 0 | −50.88 | −52.65 |

A significant increase occurred in the synchronization of the two lungs following administration of the bronchodilator as indicated by a very negative Δ( Δ̅_{τ}) (−0.75). On the basis of this observation, the case was diagnosed as asthma, and this diagnosis was confirmed by spirometery and case history.

### Case 2:

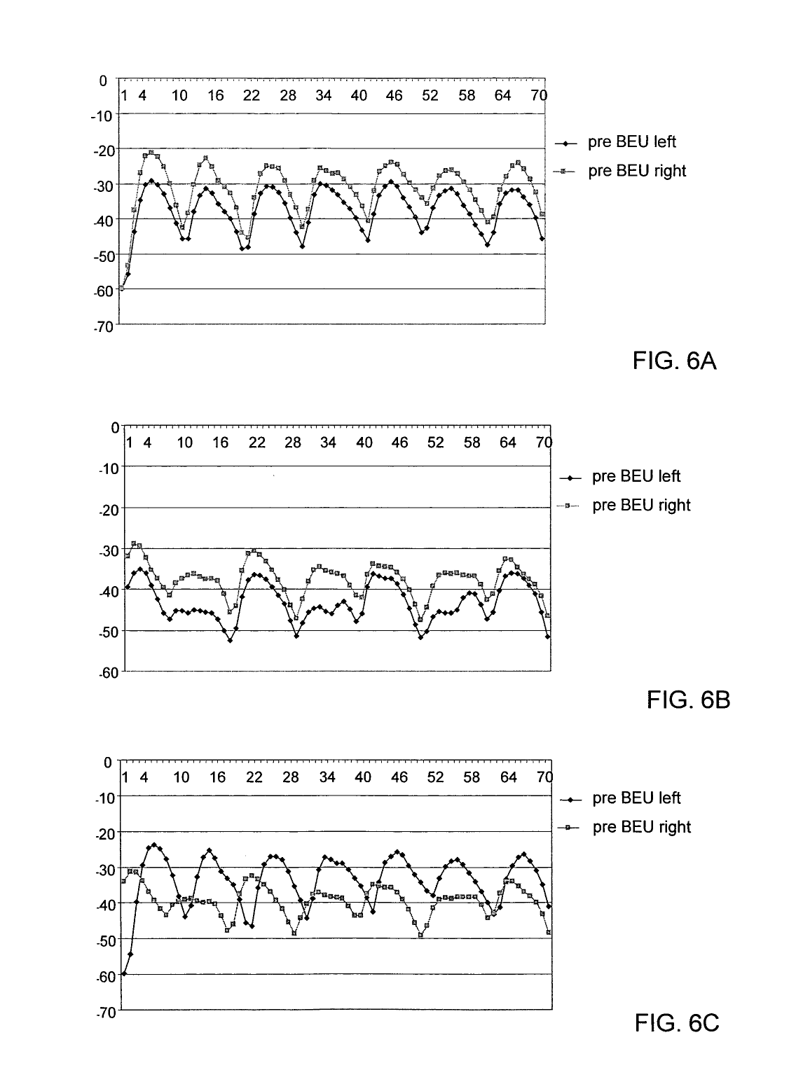

FIG. 6a shows the representative signal obtained as above for the left lung (curve a) and the right lung (curve b) of a subject obtained prior to administration of the bronchodilator. FIG. 6b shows the representative signal obtained as above for the left lung (curve a) and the right lung (curve b) of a subject obtained after administration of the bronchodilator. FIG. 6c shows the average acoustic level in decibels of both lungs before (curve a) and after (curve b) administration of the bronchodilator. The results obtained for this case are summarized in Table 2.

TABLE 2

| | Δ̅τ0 | Δ̅τ1 | h₀ | h₁ |
|---|---|---|---|---|
| | [# of frames] | [# of frames] | [db] | [db] |
| Patient # 23 | 0 | 1.43 | −33.54 | −39.37 |

A significant decrease occurred in the synchronization of the two lungs following administration of the bronchodilator as indicated by a very positive Δ( Δ̅_{τ}) (1.43). On basis of this observation, the case was diagnoses as COPD, and this diagnosis was confirmed by spirometery and case history.

### Case 3:

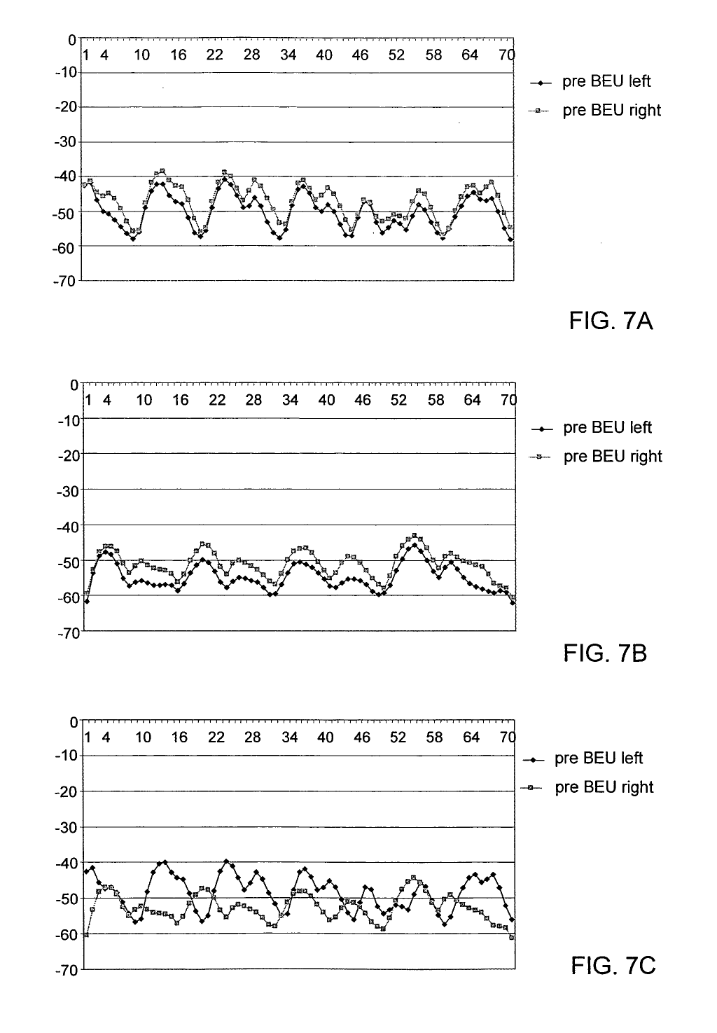

FIG. 7a shows the representative signal obtained as above for the left lung (curve a) and the right lung (curve b) of a subject obtained prior to administration of the bronchodilator. FIG. 7b shows the representative signal obtained as above for the left lung (curve a) and the right lung (curve b) of a subject obtained after administration of the bronchodilator. FIG. 7c shows the average acoustic level in decibels of both lungs before (curve a) and after (curve b) administration of the bronchodilator. The results obtained for this case are summarized in Table 3.

TABLE 3

| | Δ̅τ0 | Δ̅τ1 | h₀ | h₁ |
|---|---|---|---|---|
| | [# of frames] | [# of frames] | [db] | [db] |
| Patient # 30 | 0 | 0 | −48.35 | −52.68 |

In this case, there no change was observed in Δτ (Δ( Δ̅_{τ})=0). However, a decrease was observed in Δh. A diagnosis of COPD was therefore made which was confirmed by spirometery and case history.

### Case 4:

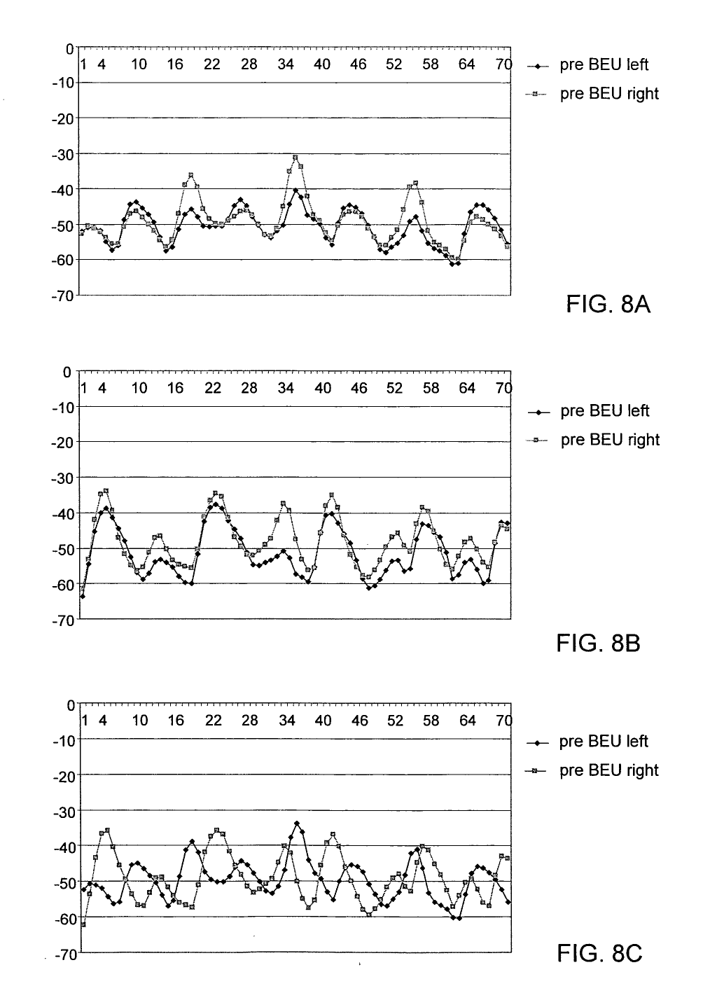

FIG. 8a shows the representative signal obtained as above for the left lung (curve a) and the right lung (curve b) of a subject obtained prior to administration of the bronchodilator. FIG. 8b shows the representative signal obtained as above for the left lung (curve a) and the right lung (curve b) of a subject obtained after administration of the bronchodilator. FIG. 8c shows the average acoustic level in decibels of both lungs before (curve a) and after (curve b) administration of the bronchodilator. The results obtained for this case are summarized in Table 4.

TABLE 4

| | Δ̅τ0 | Δ̅τ1 | h₀ | h₁ |
|---|---|---|---|---|
| | [# of frames] | [# of frames] | [db] | [db] |
| Patient # 7 | 0 | 0 | −49.51 | −49.03 |

In this case the synchronization of the two lungs as well as the value of h were unchanged by the administration of the bronchodilator. A diagnosis of COPD was therefore made which was confirmed by spirometery and case history.

### Case 5:

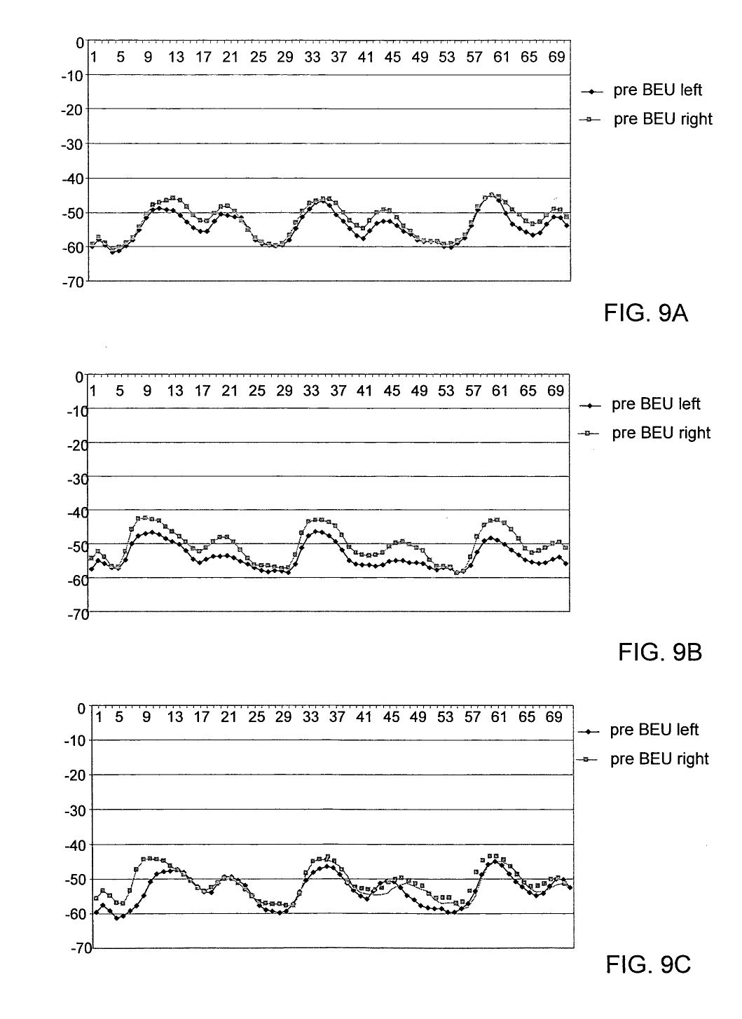

FIG. 9a shows the representative signal obtained as above for the left lung (curve a) and the right lung (curve b) of a subject obtained prior to administration of the bronchodilator. FIG. 9b shows the representative signal obtained as above for the left lung (curve a) and the right lung (curve b) of a subject obtained after administration of the bronchodilator. FIG. 9c shows the average acoustic level in decibels of both lungs before (curve a) and after (curve b) administration of the bronchodilator. The results obtained for this case are summarized in Table 5.

TABLE 5

| | Δ̅τ0 | Δ̅τ1 | h₀ | h₁ |
|---|---|---|---|---|
| | [# of frames] | [# of frames] | [db] | [db] |
| Patient # 66 | 0.67 | 0.67 | −53.27 | −51.76 |

In this case, the synchronization of the two lungs remained unchanged, and the value of h increased following administration of the bronchodilator. Before administration of the bronchodilator, the two lungs were unsynchronized. A diagnosis of COPD was therefore made which was confirmed by spirometery and case history.

### Case 6:

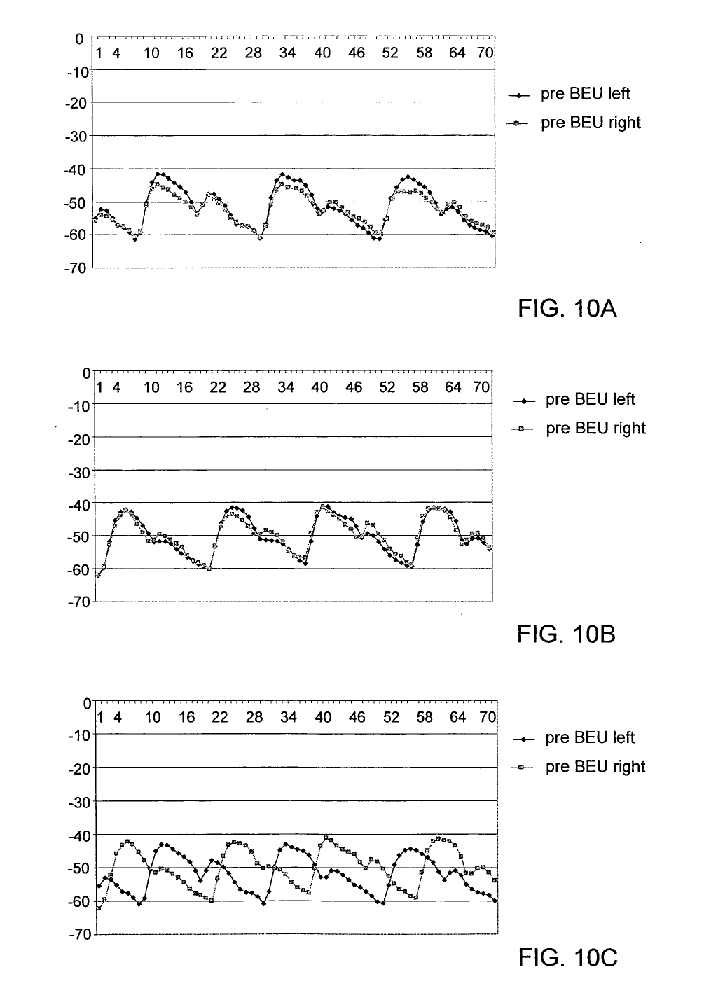

FIG. 10a shows the representative signal obtained as above for the left lung (curve a) and the right lung (curve b) of a subject obtained prior to administration of the bronchodilator. FIG. 10b shows the representative signal obtained as above for the left lung (curve a) and the right lung (curve b) of a subject obtained after administration of the bronchodilator. FIG. 10c shows the average acoustic level in decibels of both lungs before (curve a) and after (curve b) administration of the bronchodilator. The results obtained for this case are summarized in Table 6.

TABLE 6

| | Δ̅τ0 | Δ̅τ1 | h₀ | h₁ |
|---|---|---|---|---|
| | [# of frames] | [# of frames] | [db] | [db] |
| Patient # 9 | 0 | 0 | −52.00 | −49.91 |

In this case the synchronization of the two lungs remained unchanged, and the value of h increased following administration of the bronchodilator. Before administration of the bronchodilator, the two lungs were synchronized. A diagnosis of asthma was therefore made which was confirmed by spirometery and case history.

1.-42. (canceled)

43. A system for making a differential diagnosis between two respiratory tract disorders, comprising:
(a) an integer N of transducers, each transducer configured to be fixed on a surface of the individual over the thorax, the ith transducer being fixed at a location xᵢ and generating a signal Z(xᵢ,t) indicative of pressure waves at the location xᵢ; i=1 to N at times t during a predetermined time interval;
(b) a processor configured to:
receive the signals Z(xᵢ,t) and to process the signals, wherein the processing comprises performing at least one event search; determine values of one or more initial event parameters; determine values of the one or more final event parameters and
compare the values of the initial event parameters to the final event parameters, and to make differential diagnosis based on the comparison.

44. The system according to claim 43, wherein an event search is performed on one or more of the signals Z(xᵢ,t).

45. The system according to claim 43, wherein an event search is performed on one or more signals P(xᵢ,t) wherein the signals P(xᵢ,t) are obtained after performing one or more procedures on one or more of the signals Z(xᵢ,t) selected from filtering, denoising, smoothing, envelope extraction, applying a mathematical transformation.

46. The system according to claim 43, wherein the transducers are divided into one or more subsets and the processing comprises, for each of one or more of the subsets, calculating a representative signal from one or more of the signals Z(xᵢ,t) or P(xᵢ,t) obtained from transducers in the subset and performing one or more event searches on one or more of the representative signals.

47. The system according to claim 43, wherein one or more of the events are selected from an entire breathing cycle, an inspiratory phase of a breathing cycle, and an expiratory phase of a breathing cycle.

48. The system according to claim 47, wherein the representative signal of a transducer subset is a summation or an average signal of the signals obtained by the transducers in the subset.

49. The system according to claim 43, wherein the event search comprises performing any one or more of a peak search, an autocorrelation, a cross correlation with a predetermined function, and a Fourier transform.

50. The system according to claim 43, wherein one or more of the event parameters are selected from the group consisting of a time that an event occurred, a duration of an event, a magnitude of an event, a height of a peak associated with the event, the width of a peak associated with the event in a signal at half peak height, a half time to rise of a peak associated with the event in a signal, a half time to fall of a peak, an area under a peak; a maximum of the signal during the event, a ratio of a maximum during an inspiratory phase to a maximum during an expiratory phase, a ratio of a duration of an inspiratory phase to a duration of an expiratory phase, and a morphology of a signal during the event.

51. The system according to claim 43, wherein the processor is further configured to calculate one or more comparisons between an event parameter value and a predetermined threshold or range of values.

52. The system according to claim 46, wherein the processor is further configured, for each of one or more pairs of a first representative signal and a second representative signal, to calculate one or more comparisons between an event parameter value calculated for the first representative function and an event parameter value calculated for the second representative function.

53. The system according to claim 51, wherein the processor is further configured to make a diagnosis based upon one or more of the comparisons.

54. The system according to claim 43, wherein the transducers are divided into one or more sets, and an event parameter is a time at which an event occurred in a representative signal of each set and the comparison involves determining an extent of synchrony between two signals.

55. The system according to claim 43, wherein an event parameter is an average magnitude of a signal over a time period.

56. The system according to claim 43, wherein the processor is configured to diagnose asthma on the basis of the comparison.

57. The system according to claim 43, wherein the processor is configured to diagnose COPD on the basis of the comparison.

58. The system according to claim 43, further comprising a display device.

59. The system according to claim 58, wherein the processor if further configured to display on the display device a result of a calculation, diagnosis, or determination made by the processor.

60. The system according to claim 51, wherein the processor is configured to make a differential diagnosis of COPD and asthma wherein :
(a) the one or more initial event parameters are:
(i) an initial mean value h of the signal over the predetermined time interval, h₀, calculated for a representative signal obtained on a first subset of transducers prior to administration of a bronchodilator; and
(ii) an initial time delay, Δ̅_{τ0}, between a time of a peak in a signal calculated for a second transducer set and a time of a corresponding peak calculated for a third transducer set prior to administration of the bronchodilator;
(b) the one or more final event parameters are:
(i) a final h, h₁, calculated for a representative signal obtained on the first subset of transducers after administration of the bronchodilator; and
(ii) an final time delay, Δ̅_{τ}_{₁}, between a time of a peak in a signal calculated for a second transducer set and a time of a corresponding peak calculated for a third transducer set prior to administration of the bronchodilator;
and wherein the processing comprises:
i) calculating a change in h, Δh, where Δh=h₁−h₀;
ii) calculating a change in Δ̅_{τ}, Δ( Δ̅_{τ}), where Δ( Δ̅_{τ})= Δ̅_{τ1}− Δ̅_{τ0};
iii) making a differential diagnosis of COPD if Δ( Δ̅_{τ})>d₁, where d₁ is a predetermined first threshold;
iv) making a differential diagnosis of asthma if (i) Δ( Δ̅_{τ})≦d₁; and if (ii) Δ( Δ̅_{τ})<−d₁;
v) making a differential diagnosis of COPD if (i) |Δ( Δ̅_{τ})|<d₁, and if (ii) Δh≦0;
vi) making a differential diagnosis of COPD if (i) Δh≧0, and if (ii) Δ̅_{τ0>d2}, where d2 is a predetermined second threshold; and
vii) making a differential diagnosis of asthma if (i) Δh≧0, and if (ii) Δ̅_{τ0}≦d₂.

61. A method for making a differential diagnosis between two respiratory tract disorders comprising:
(a) obtaining an integer N of signals Z(xᵢ,t) indicative of pressure waves at locations xᵢ; for i=1 to N over the thorax at times t during a predetermined time interval;
(b) processing the signals Z(xᵢ,t), wherein the processing comprises performing at least one event search;
(c) determining values of one or more initial event parameters;
(d) determining values of the one or more final event parameters; and
(e) comparing the values of the initial event parameters to the final event parameters to make the differential diagnosis.

62. The method according to claim 61, wherein an event search is performed on one or more of the signals Z(xᵢ,t).

63. The method according to claim 61, wherein an event search is performed on one or more signals P(xᵢ,t) wherein the signals P(xᵢ,t) are obtained after performing one or more procedures on one or more of the signals Z(xᵢ,t) selected from filtering, denoising, smoothing, envelope extraction, applying a mathematical transformation.

64. The method according to claim 61, wherein the transducers are divided into one or more subsets and the processing comprises, for each of one or more of the subsets, calculating a representative signal from one or more of the signals Z(xᵢ,t) or P(xᵢ,t) obtained from transducers in the subset and performing one or more event searches on one or more of the representative signals.

65. The method according to claim 61, wherein one or more of the events are selected from an entire breathing cycle, an inspiratory phase of a breathing cycle, and an expiratory phase of a breathing cycle.

66. The method according to claim 65, wherein the representative signal of a transducer subset is a summation or an average signal of the signals obtained by the transducers in the subset.

67. The method according to claim 61, wherein the event search comprises performing any one or more of a peak search, an autocorrelation, a cross correlation with a predetermined function, and a Fourier transform.

68. The method according to claim 61, wherein one or more of the event parameters are selected from the group consisting of a time that an event occurred, a duration of an event, a magnitude of an event, a height of a peak associated with the event, the width of a peak associated with the event in a signal at half peak height, a half time to rise of a peak associated with the event in a signal, a half time to fall of a peak, an area under a peak; a maximum of the signal during the event, a ratio of a maximum during an inspiratory phase to a maximum during an expiratory phase, a ratio of a duration of an inspiratory phase to a duration of an expiratory phase, and a morphology of a signal during the event.

69. The method according to claim 61, further comprising calculating one or more comparisons between an event parameter value and a predetermined threshold or range of values.

70. The method according to claim 64, further comprising, for each of one or more pairs of a first representative signal and a second representative signal, calculating one or more comparisons between an event parameter value calculated for the first representative function and an event parameter value calculated for the second representative function.

71. The method according to claim 61, further comprising performing a medical treatment of the individual after determining the initial event parameters.

72. The method according to claim 71, wherein the medical treatment comprises administering a bronchodilator.

73. The method according to claim 61, wherein the transducers are divided into one or more sets, and an event parameter is a time at which an event occurred in a representative signal of each set and the comparison involves determining an extent of synchrony between two signals.

74. The method according to claim 61, wherein an event parameter is an average magnitude of a signal over a time period.

75. The method according to claim 61, further comprising diagnosing asthma on the basis of the comparison.

76. The method according to claim 61, further comprising diagnosing COPD on the basis of the comparison.

77. The method according to claim 61, wherein the differential diagnosis is a differential diagnosis of COPD and asthma wherein :
(a) the one or more initial event parameters are:
(i) an initial the mean value h of the signal over the predetermined time interval, h₀, calculated for a representative signal obtained on a first subset of transducers prior to administration of a bronchodilator; and
(ii) an initial time delay, Δ̅_{τ}_{₀}, between a time of a peak in a signal calculated for a second transducer set and a time of a corresponding peak calculated for a third transducer set prior to administration of the bronchodilator;
(b) the one or more final event parameters are:
(i) a final h, h₁, calculated for a representative signal obtained on the first subset of transducers after administration of the bronchodilator; and
(ii) an final time delay, Δ̅_{τ}_{₁}, between a time of a peak in a signal calculated for a second transducer set and a time of a corresponding peak calculated for a third transducer set prior to administration of the bronchodilator;
and wherein the method comprises:
(a) calculating a change in h, Δh, where Δh=h₁−h₀;
(b) calculating a change in Δ̅_{τ}, Δ( Δ̅_{τ}), where Δ( Δ̅_{τ})= Δ̅_{τ1}− Δ̅_{τ0};
(c) making a differential diagnosis of COPD if Δ( Δ̅_{τ})>d₁, where d₁ is a predetermined first threshold;
(d) making a differential diagnosis of asthma if (i) Δ( Δ̅_{τ})≦d₁; and if (ii) Δ( Δ̅_{τ})<−d₁;
(e) making a differential diagnosis of COPD if (i) |Δ( Δ̅_{τ})|<d₁, and if (ii) Δh≦0;
(f) making a differential diagnosis of COPD if (i) Δh≧0, and if (ii) Δ̅_{τ0>d2}, where d2 is a predetermined second threshold; and
(g) making a differential diagnosis of asthma if (i) Δh≧0, and if (ii) Δ̅_{τ0}≦d₂.

78. The system according to claim 52, wherein the processor is further configured to make a diagnosis based upon one or more of the comparisons.
